(19) United States
(12) Patent Application Publication
(10) Pub. No.: US 2016/0184553 A1
(43) Pub. Date: Jun. 30, 2016

(54) A PLASTER DEVICE

(71) Applicant: LINA MEDICAL APS, Glostrup (DK)

(72) Inventor: Philip FLEISCHER, Copenhagen S (DK)

(21) Appl. No.: 14/406,433

(22) PCT Filed: Jun. 7, 2013
(86) PCT No.: PCT/EP2013/061836

§ 371 Date: Dec. 8, 2014

Related U.S. Application Data
(60) Provisional application No. 61/657,592, filed on Jun. 8, 2012.

(30) Foreign Application Priority Data
Jun. 8, 2012 (DK) PA2012 70311
Dec. 6, 2012 (DK) PA201270764

(51) Int. Cl. A61M 25/02

(52) U.S. Cl. CPC A61M 25/02; A61M 2025/026; A61M 2025/0266

(57) ABSTRACT

The invention provides a plaster device for securing a medical tube to a skin surface of an individual having the tube inserted into a body part via an access opening. The plaster device comprises a plaster having a lower surface which is at least partly adhesive and facilitates attaching the plaster device to the skin. The plaster further has an opposite upper surface which comprises at least one strap for strapping a length of the tube to the upper surface of the plaster.

### FIELD OF THE INVENTION

The present invention relates to a plaster device for securing a medical tube to a skin surface of an individual having the tube inserted into a body part via an access opening.

### BACKGROUND OF THE INVENTION

A central venous catheter is a tube placed into a large vein in the neck, chest, arm, or groin. It is used to administer medication or fluids, obtain blood tests, and directly obtain cardiovascular measurements e.g. related to the central venous pressure etc.

The location of the vein is typically identified with the use of a small ultrasound device. A hollow needle is advanced through the skin until blood is aspirated, and ultrasound is used during insertion to monitor the insertion and to ensure correct positioning of the tube.

Non-tunnelled catheters are fastened at the site of insertion, with the tube and attachments protruding directly. Tunnelled catheters are passed under the skin from the insertion site to a separate exit site where the catheter and its attachments emerge from underneath the skin. The exit site is typically located in the chest, making the access port less visible than if the catheter were to directly protrude from the neck. Passing the catheter under the skin helps to prevent infection and provides stability.

Depending on its use, the tube is mono-luminal, bi-luminal or tri-luminal, dependent on the actual number of lumens (1, 2 and 3 respectively). Some tubes have 4 or 5 lumens, depending on the reason for their use.

It is important that the position of the tube inside the body remains over time. The tube is usually held by an adhesive dressing, suture, or by a staple which is covered by an occlusive dressing. Sometimes, however, it is desirable to enable axial movement of the tube for exact positioning in the body, and the traditional ways of fastening the tube by suture or staple is typically not flexible with regards to axial re-positioning.

### DESCRIPTION OF THE INVENTION

It is an object of embodiments of the invention to provide an improved plaster device for securing a medical tube to a skin surface of an individual having the tube inserted into a body part via an access opening.

It is a further object of embodiments of the invention to provide a plaster device for securely fastening of the tube and yet allowing axial re-positioning of the tube, and thus allowing temporary fastening of the tube during insertion, e.g. for maintaining a preliminary position of the tube until a correct position is verified by ultrasound, etc.

It is an even further object of embodiments of the invention to provide a plaster device which can be positioned more freely relative to the access opening such that a fixed distance between the access opening and the plaster device not necessarily need to be observed.

According to a first aspect, the invention provides a plaster device for securing a medical tube to a skin surface of an individual having the tube inserted into a body part via an access opening, the plaster device comprising a plaster having a lower surface which is at least partly adhesive and facilitates attaching the plaster device to the skin, and an opposite upper surface, wherein the device further comprises at least one strap for strapping a length of the tube to the upper surface of the plaster.

By “strapping”, is herein meant that the strap can be wrapped at least above the tube such that the tube becomes securely fastened between the upper surface of the plaster and the strap. Strapping may also include that the strap is wrapped completely around the tube in question, or even that it is curled several times around the tube.

The “strap” may include any kind of elongate element such as a string, wire, band, or tape, etc., i.e. any elongated structure which can be wrapped at least partly around the tube. The strap may have a laminated structure obtained by joining several layers of identical or different materials.

The lower surface of the plaster, which is at least partly adhesive, facilitates attachment of the plaster device to the skin. In an alternative embodiment, the entire lower surface may be adhesive.

Either before or after the tube is inserted into the access opening, the plaster device is attached to the skin by use of the at least partly adhesive lower surface. Typically, the plaster device is located close to an access opening.

If the plaster device is attached prior to the insertion of the tube, the physician may use the plaster device for supporting the tube during insertion, or the strap may provide temporary fastening of the tube during insertion.

If the plaster device is attached after the tube has been inserted, the physician may use the strap for preliminary fastening while the exact position of the tip is verified by ultrasound, or for permanent fastening of the tube. In connection with preliminary fastening, the strap of the plaster device enables adjustment of the tube position, e.g. by unstrapping the tube or by loosening the strap until the correct position is obtained.

To improve fastening of the tube further, and thus retain the tube at a fixed position after insertion, e.g. to avoid injuries and discomfort, the strap may include a layer of a material exposing a high frictional resistance against sliding of the tube against an inner surface of the strap. As aforementioned, the strap may include a layered structure where one layer exposes a higher resistance against sliding of the tube than the other layers of the strap.

In order to further improve fastening of the tube and to retain the tube at a fixed position, the upper surface of the plaster may further comprise an adhesive surface portion for retaining a fixed position of the tube relative to the upper surface. The tube may be attached to the adhesive surface portion of the upper surface by pressing it onto this surface portion.

The adhesive surface portion of the upper surface may be constituted by a portion of the plaster which is folded such that a portion of the adhesive lower surface forms part of the upper surface.

To further facilitate secure fastening of the tube after final positioning, and to provide a shield for the access opening, the plaster device may comprise a cover connected to, or formed in one part with the plaster. The cover is formed with a lower surface which is at least partly adhesive and sufficiently elastically deformable to facilitate attachment to the surface of the skin to cover the skin, the access opening, and a part of the tube.

In one embodiment of the plaster device, the cover and the plaster is formed in one piece.

To increase comfort, the cover may be more elastically deformable than the plaster. The larger elastic deformability of the cover as compared to the plaster facilitates that the cover can be easily shaped around the tube and thereby forms a sealing cover for the access opening. If the cover is shaped tightly around the tube, the risk of infections can be reduced, and the elasticity further increases comfort by allowing a higher degree of freedom for the skin surface to move. By “larger elastic deformability” is herein meant that it is stretchable and/or foldable by use of less force, and/or it can be deformed elastically to a larger extend before the deformation becomes plastic and thus permanent.

To enable visual inspection of the skin and access opening, e.g. to watch for incipient infection, the cover may be transparent, and particularly, it may comprise different layers, e.g. comprising a transparent PU film layer and a transparent layer of an adhesive, e.g. a transparent hydrocolloid or silicone adhesive layer.

At least that part of the plaster device which, in use, comes in direct contact with the skin could be made from hydrocolloid or a similar skin-friendly material. This applies e.g. to the lower surface of the plaster and the lower surface of the cover.

To avoid skin irritation, the adhesive of the lower surface of the plaster and the lower surface of the cover may be selected from the group consisting of hydrocolloid adhesive, medical acrylic adhesive, Silicone adhesive, Hydrogel adhesive, Hot-melt adhesive, Polyurethane adhesive, PVA adhesive, and other skin-friendly adhesives which are considered safe for use in contact with the skin.

To protect the adhesive of the lower surface of the plaster before use, the plaster may comprise a removable protective layer. The protective layer may be made from siliconized paper, siliconized plastic film or foil, such as PET, PP, PE, PA, or other plastic sheet material. The protective layer may be relatively thin and flexible, such as less than 100 micron, or even less than 50 micron.

The optional adhesive surface portion of the upper surface of the plaster for retaining a fixed position of the tube relative to the upper surface may comprise e.g. a PU adhesive, a PVA adhesive, or a rubber adhesive. A similar adhesive may be applied between layers of the plaster device as this adhesive should not come in contact with the skin.

In the transition between the interconnected cover and plaster, the plaster device may form a hinge-structure allowing rotation of the cover relative to the plaster about a line, particularly about a straight line. The hinge structure may be constituted by a weakened line, e.g. a depression being embossed into the material or a dotted perforation of the material in the transition between the cover and plaster.

The lower surface of the cover and the lower surface of the plaster may thus be oppositely arranged, so that the lower surface of the cover is facing away from the skin before attachment of the cover to the skin. After final positioning of the tube, the cover is rotated about the straight line hinge and the adhesive lower surface of the cover thereby faces towards the skin. Due to the interconnection and the hinge-structure, the cover thereby becomes easy to position once the plaster is positioned, and the physician can therefore concentrate the effort on establishing a completely sealing bond between the cover, the skin, and the tube across the access opening.

In one embodiment, the cover is formed such that its adhesive lower surface comes in contact with the optional adhesive upper surface portion of the plaster. By covering the adhesive surface portion of the upper surface, it may be achieved that e.g. clothing, hair, dirt, and other things, do not get adhered to this adhesive surface portion, and the contact between two adhesive surfaces creates a superior bond between the cover and plaster and thereby a strong retention of the tube there between.

The adhesive surface portion of the upper surface and the adhesive of the lower surface of the cover may be engineered to match each other in different ways. In one embodiment, they may be non-releasable whereby only destructive disassembly and removal of the tube is possible, or they may be engineered for releasable bonding such that the catheter tube can be released without destruction of the plaster device.

The cover, when attached to the skin and covering the access opening, co-extends the plaster in one direction. Herein, we refer to this direction as “a longitudinal support direction for the tube”. Typically, the tube will extend in this direction when located in the device.

The at least one strap may be adapted for strapping a tube which is arranged in the longitudinal support direction. Accordingly, the strap may particularly extend transversely or perpendicularly to the longitudinal support direction.

In one embodiment, the cover may have a smaller surface area than the plaster. However, depending on the use of the tube, the surface area of the cover may, in an alternative embodiment, have a larger surface area than the plaster. Generally, the cover may advantageously be smaller than the plaster since the plaster would typically be applied very close to the access opening and the cover is primarily for covering that access opening. Accordingly, the total size of the device may be reduced by making the cover smaller than the plaster, e.g. with a surface area in the range of 25-75 percent of the area of the plaster.

To facilitate a closed-loop configuration of the strap around the tube, the device may further comprise a head portion formed with a slot which has a size whereby one of the at least one straps can be received through the slot. In use, the free end of the strap can be inserted through the slot, folded backwards, and attached to the upper surface, e.g. adhesively, or it can be tied to other straps of the device.

Also to facilitate the closed-loop configuration, the strap may be a double-sided strap having opposite first and second surfaces with cooperating surface structures such that the opposite surfaces can be joined. This enables a loop configuration when one surface of the strap is joined to the other surface of the same strap after the strap has been wrapped around the tube. The aforementioned head portion may, likewise be double-sided.

In one embodiment, at least one strap, herein referred to as a binder is double-sided and forms in one free end a head of the kind already described, i.e. with a slot. In an opposite free end, the binder forms a tail portion which is a free end which can be received in the slot of the head of the same binder or of another binder of the device to thereby form the closed loop.

The head with the slot makes it particularly easy to tighten the closed loop about the tube, particularly if the strap forms a tab or free end adjacent the through hole where the user can grip the strap. In this embodiment, the tube is attachable to the plaster device as follows: a) the tube is positioned relative to the plaster device—typically in the longitudinal support direction, b) strap is curled around the tube and the tail portion is inserted through the slot, c) the tab shaped free end and the tail portion are pulled tightly to tighten the strap about the tube, d) the first surface is pressed gently against the second surface whereby the cooperating surface features engage and the loop-shape is closed. In the following, we will use the term “head and tail configuration” for describing the configuration of the strap with a through hole in the head for receiving a tail portion and provide easy tightening around the tube.

To facilitate easy and releasable strapping of the tube, the at least one strap may comprise a strapping structure facilitating releasable fastening of the strap to itself, to another strap, or to a portion of the plaster. Such a strapping structure may include a hook-and-loop type fastening structure of the kind known e.g. from Velcro™. This kind of fastener is characterised by its ability to join to elements by pressing them together and to allow the elements to be released from each other by pulling them away from each other. In this structure a hook surface comprising projecting hook-like structures and a cooperating loop surface comprising loop-like structure are joined by engagement between the hooks and loops, i.e. they become entangled. Herein, we use the term hook-and-loop type releasable fastener to cover any kind of such entanglement based fastener irrespective of the exact shape of the hooks and loops. The strapping structure may include other means for mechanical fixation, e.g. buttons, projections received in holes etc., or it may include an adhesive.

If the strap or the head is double-sided, the hook surface may be on one side of the strap and the loop surface may be on an opposite side of the strap. Alternatively, or in combination with the hook-and-loop fastener structure, the strap may include an adhesive surface, particularly one allowing releasable fastening, e.g. an adhesive allowing repeated joining and release of the strap.

In the combination between adhesive and hook-and-loop fastener structure, either the hook-surface, or the loop-surface, or both surfaces may include an adhesive. In this embodiment, the strap may adhere to another surface irrespective if the other surface has a matching hook or loop structure. This feature may advantageously apply e.g. to prevent free ends from extending outwards and thereby to prevent flaps which can become caught by clothes etc.

In yet another embodiment, the strap is flexible enough to facilitate binding or tying of the strap.

The at least one strap may be made form a combined hook & loop material such as made from PA and PP, or other plastic materials. As an alternative to a hook & loop material, the at least one strap may be made from non-woven materials, such as made from PA, PP, PET, or other plastic materials. The strap may thus comprise an adhesive layer, e.g. comprising an acrylic adhesive, a hot-melt adhesive, PU adhesive, or other adhesives. The adhesive properties may be combined with the hook & loop material. As a further alternative, the at least one strap may be made from a plastic material tape with adhesive, a non-woven material without adhesive for straps to be joined by a knot.

To facilitate axial re-positioning of the tube without transverse displacement hereof, the plaster device may comprise at least two sets of straps arranged at a distance from each other such that the tube is passed between two fastening points. This may effectively fasten the catheter and simultaneously provide support for linear movement of the tube between the two sets of straps. This may be further facilitated by arranging the at least two straps at a distance in the longitudinal support direction, e.g. a distance in the range of 50-75 percent of the dimension of the plaster in the longitudinal support direction.

The plaster may be formed with a laminate structure comprising a number of layers which are stacked and joined adhesively. In this case, the at least one strap may be located between a base which forms the lower surface and a top which forms the upper surface.

The base and top may be joined adhesively on opposite sides of the at least one strap. However, the at least one strap and possible other layers may be arranged between the base and the top, as the plaster may comprise a plurality of layers.

Since the base forms the lower surface of the plaster, the base may particularly be made from any of the previously mentioned materials suitable for the lower surface to avoid skin irritation, e.g. from hydrocolloid, silicone, PU, PVA or combinations thereof.

The base and cover may be in one part—i.e. the base and cover may be made from a single sheet of a skin friendly material. In this embodiment, the aforementioned hinge-structure in the transition between the interconnected plaster and cover could be formed e.g. by pre-folding or by scoring a mark in the sheet such that it folds easily along this mark.

One layer could be a foam layer which may have a thickness of at least half the total thickness of the plaster. The foam layer may form a protective layer shielding the person from the pressure of the tube. Particularly, the foam layer may be arranged on top of the layered structure and thereby form at least a part of the upper surface of the plaster.

The at least one strap may be constituted by one single layer in the laminate structure. If the plaster device comprises e.g. 2, 3, 4 or 5 straps, such straps may preferably be in the form of elongated projections in a single piece of a sheet material which forms one of the layers in in the laminate structure. Herein we refer to this layer as the “strap layer”. The sheet material could e.g. be entirely made from a hook & loop fastener material which is cut into the mentioned shape with elongated projections forming each of the straps.

The strap layer may particularly be located between the base and a layer of a non-woven material.

The foam layer may comprise a section having a thickness which is tapered down towards an edge of the foam layer, whereby the thickness of the foam layer may be thinner at one end. The foam layer, and thus the plaster device, may be positioned so that the thickness of the foam layer tapers down towards the access opening as this may provide a better support for the tube, whereby undesirable bending of the tube may be avoided. To form the tapered shape of the foam layer, the foam may comprise indentations formed in one serrated edge. By such indentations, the foam layer becomes more easily compressed at the serrated edge.

In one embodiment, the foam is arranged on top of the layered structure and therefore forms part of the upper surface of the plaster. To compress the serrated edge and thereby form the mentioned tapered shape, an edge portion of the base is folded to cover the serrated edge of the foam. Simultaneously, the adhesive lower surface of this folded portion of the base thereby forms the adhesive upper surface portion of the plaster and thereby facilitates better fastening of the tube to the upper surface. I.e. the plaster device may comprise a tapered portion with a layered structure including a serrated foam material and a layer which compresses the foam material.

In one embodiment, the at least one strap may be positioned between the foam layer and the base. The foam layer may e.g. comprise closed cell foam made from PE, PU, PVC, or other plastic material. The density of the foam may be in the range of 20-70 kg/m³, such as in the range of 25-40 kg/m³.

The top may protect the lower layers, and may e.g. be made from a non-woven or other plastic tapes with adhesive in the form of PU adhesive, PVA adhesive, or rubber adhesive. Alternatively, the top is at least partly constituted by the foam.

In a second aspect, the invention provides a method of securing a medical tube to a skin surface of an individual having a tube inserted into a body part via an access opening by use of a plaster device as described above. The method comprises the steps of attaching the lower surface of the plaster device to the skin surface adjacent to the access opening, arranging the tube in a longitudinal support direction relative to the plaster device and against the upper surface of the plaster device, wrapping the strap(s) around the tube, and fixing a free end of the strap to the upper surface, to the strap itself, to a head portion, or to another strap of the same plaster device.

### BRIEF DESCRIPTION OF THE DRAWINGS

Embodiments of the invention will now be further described with reference to the drawings, in which:

FIGS. 1 and 2 illustrate two different embodiment of a plaster device according to the invention,

FIGS. 3 and 4 illustrate the plaster device of FIG. 1 attached to the skin of a person,

FIGS. 5a, 5b, and 6 illustrate exploded views of two different embodiment of a plaster device according to the invention,

FIG. 7 illustrates the attachment procedure of a plaster device according to the invention,

FIG. 8 illustrates an embodiment of the plaster device with a double-sided hook-and-loop type fastener strap having matching head and tail portions,

FIG. 9 illustrates a plaster with a strap having matching head and tails, and

FIG. 10 illustrates an exploded view of a plaster including the strap structure shown in FIG. 8.

### DETAILED DESCRIPTION OF THE DRAWINGS

It should be understood that the detailed description and specific examples, while indicating embodiments of the invention, are given by way of illustration only, since various changes and modifications within the spirit and scope of the invention will become apparent to those skilled in the art from this detailed description.

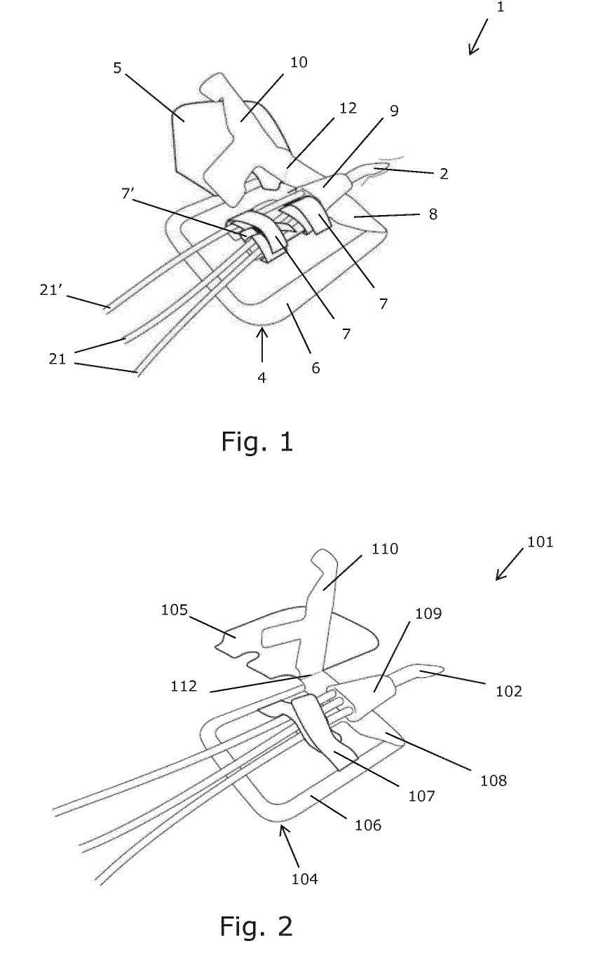

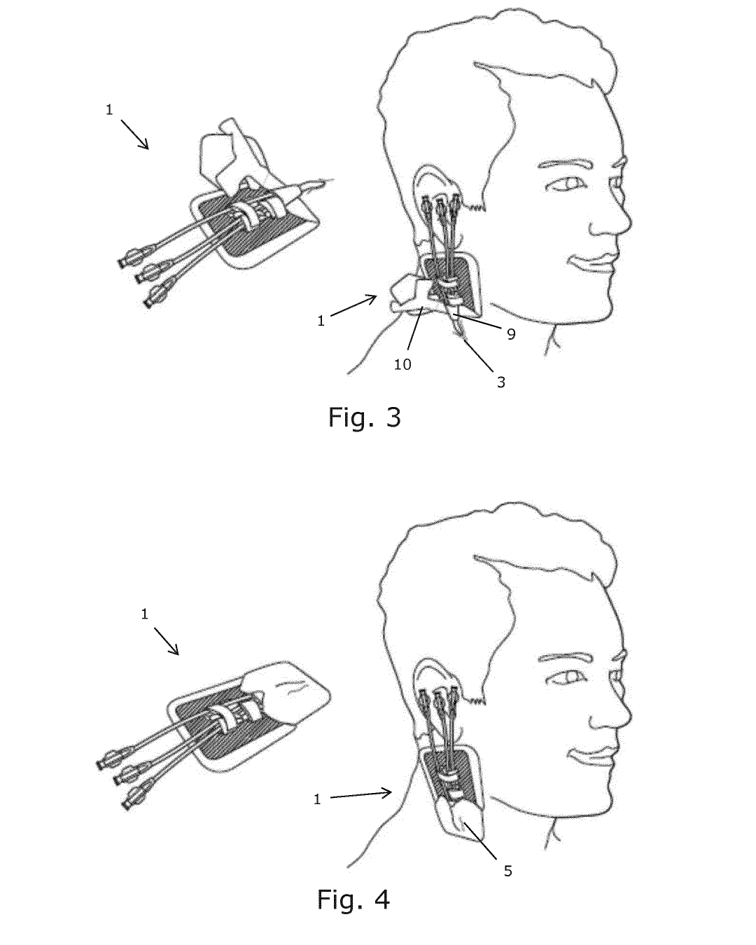

FIGS. 1 and 2 illustrate two different embodiment of a plaster device 1, 101 for securing a medical tube 2, 102 to a skin surface of an individual having the tube 2, 102 inserted into a body part via an access opening 3 (see e.g. FIG. 3). The plaster device 1, 101 comprises plaster 4, 104 and a cover 5, 105.

The plaster 4, 104 comprises a base 6, 106 having a lower surface which is at least partly adhesive and facilitates attaching the plaster device 1, 101 to the skin, and an opposite upper surface.

The upper surface comprises the plaster 4, 104 comprises at least one strap 7, 107 for strapping a length of the tube 2 to the device. In the embodiment of FIG. 1, the upper surface comprises two straps 7, whereas the embodiment of FIG. 2 comprises one strap 107 for strapping a length of the tube 2, 102 to the plaster device 1, 101.

The plaster device 101 of FIG. 2 which only comprises one strap 107 may be applicable of a child, whereas the larger plaster device 1 of FIG. 1 which comprises two straps 7 may be applicable e.g. for an adult. Furthermore, the plaster device 1 comprises an additional strap 7′ which is further adapted for separation of the lumens 21, 21′ respectively, as the additional strap 7′ is positioned substantially perpendicularly to the straps 7.

The upper surface of the plaster 4, 104 further comprises an adhesive surface portion 8, 108 for retaining a fixed position of the tube 2, 102 relative to the upper surface. The tube can be attached to the adhesive surface portion 8, 108 by pressing it onto this surface portion. In the illustrated embodiment, the junction 9, 109 of the tube is attached to the adhesive surface portion after removal of the protective layer 10, 110 which covers the adhesive surface portion before use.

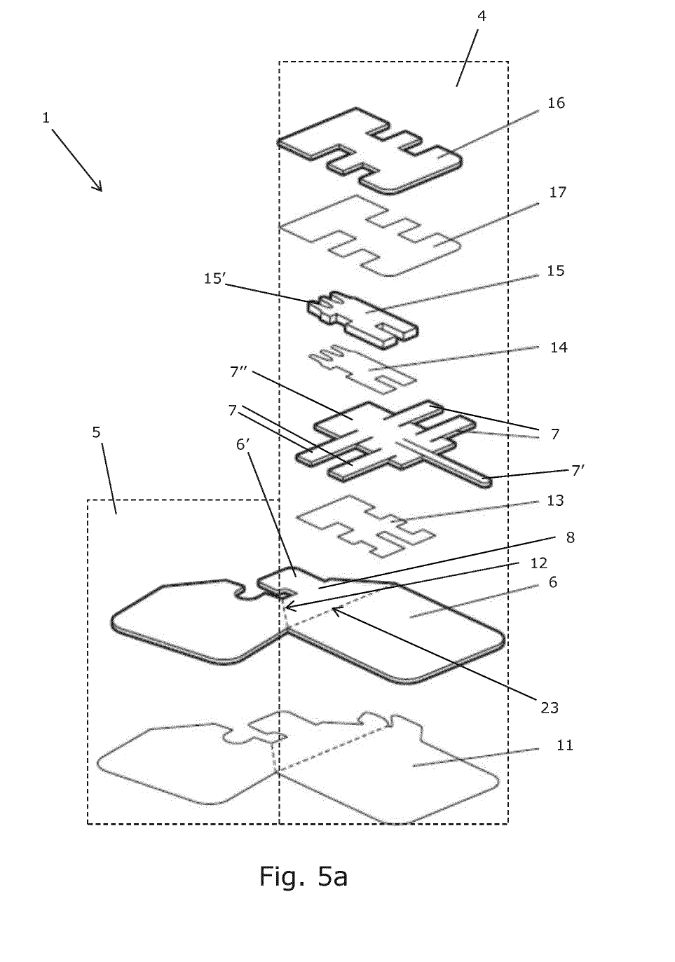

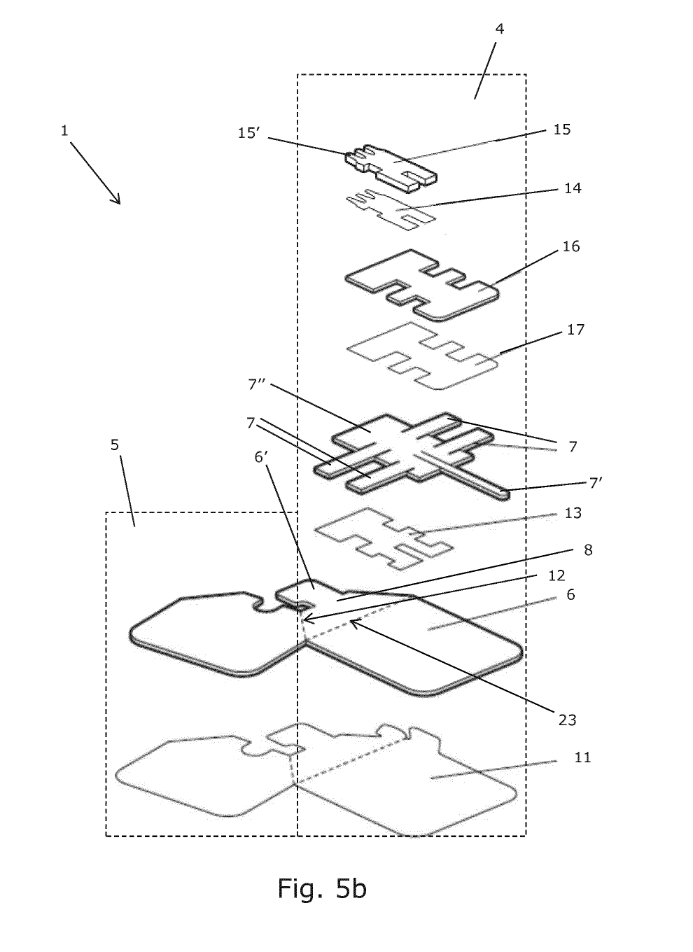

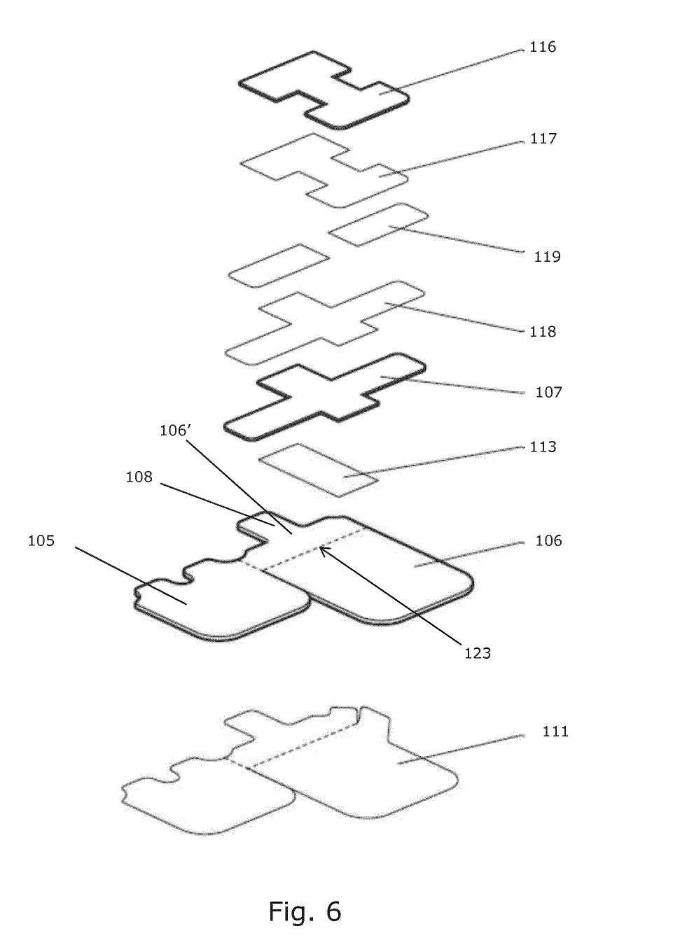

Another protective layer 11, 111 (see FIGS. 5 and 6) has covered the lower surface of the plaster 4, 104 before attaching it to the skin.

To further facilitate that the tube 2, 102 remains at the required position after final positioning hereof, the cover 5, 105 has a lower surface which is at least partly adhesive and facilitates attaching the cover to the skin.

The cover 5, 105 and the plaster 4, 104 are formed in one piece (see FIGS. 5 and 6 for more details). The cover 5, 105 and the plaster 4, 104 are interconnected, and a hinge-structure 12, 112 forms a transition there between. The hinge-structure is illustrated by the dotted line and allows rotation of the cover relative to the plaster about a straight line.

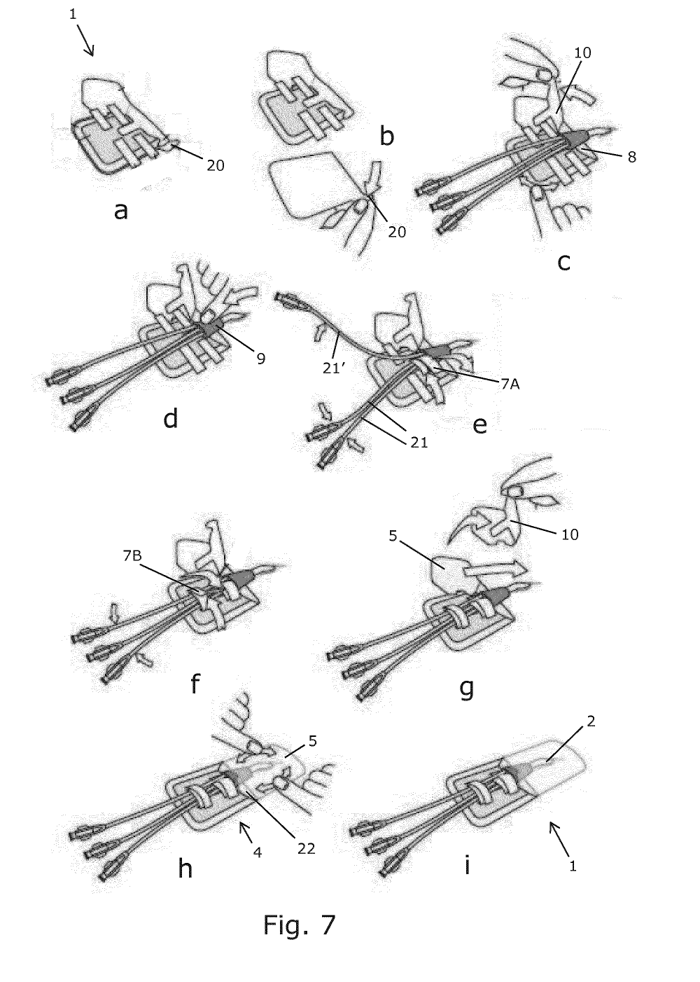

The lower surface of the cover 5, 105 and the lower surface of the plaster 4, 104 are thus oppositely arranged, so that the lower surface of the cover 5, 105 is facing away from the skin before attachment of the cover to the skin. After final positioning of the tube 2, 102, the cover can be rotated relative to the axis whereby it covers a part of the tube and thus secures the tube to the skin surface (see e.g. FIGS. 4 and 7). This rotational movement of the cover 5, 105 is carried out after the junction 9, 109 of the tube has been attached to the adhesive surface portion 8, 108 of the upper surface of the plaster 4, 104.

Accordingly, the cover 5, 105 is positioned so that is covers a part of the length of the tube 2, 102 after insertion of the distal part hereof (see FIGS. 4 and 7), when rotated relative to the straight line defined by the hinge-structure 12, 112.

The straps 7, 7′, 107 are double-sided straps comprising a hook-and-loop type structure, where the hooks are provided on one side of the strap and the loops are provided on an opposite side of the strap. This makes it very easy to join the free ends of the straps to form a closed loop around the tube. Particularly, the double-sided structure makes it possible to produce matching free strap ends in one part, i.e. being cut out from one and the same sheet of a hook-and-loop fabric, and it makes it possible to obtain bonding between the free ends by use of any one of the opposite surfaces of each of the free ends since both surface include the hook-and-loop structure.

FIGS. 3 and 4 illustrate the plaster device 1 of FIG. 1 attached to the skin of an adult. In FIG. 3, the protective layer 11 (see FIG. 7) has been removed, and the plaster 4 has been attached to the skin by the adhesive layer on the lower surface of the base 6.

The other protective layer 10 has been removed from the adhesive surface portion 8 of the upper surface of the plaster 4 and the junction 9 has been attached to the adhesive surface portion 8. The protective layer 10 also covers the lower surface of the cover 5 from which it has not been removed.

In FIG. 4, the protective layer 10 has been fully removed, and the cover 5 has been rotated by use of the hinge-structure 12 so that it covers a part of the tube 2 and the access opening 3 (see FIG. 3), and thus secures the tube 2 to the skin surface of the person.

FIGS. 5a, 5b and 6 illustrate exploded views of the two different embodiment of the plaster device 1, 101 of FIGS. 1 and 2. The plaster device 1, 101 is formed as a laminate structure comprising a number of different layers.

The plaster device 1 comprises a plaster 4 and a cover 5 as illustrated by the dotted squares. The lower layer 11 is a protective layer which is protecting the base 6 and the skin-friendly adhesive at the lower surface before use.

All straps 7, 7′ are made in one piece and forms a strap layer 7″. In this strap layer 7″, each strap forms an elongated projection. The shape is made by stamping or cutting out the desired shape from a piece of a sheet material.

An adhesive 13 is arranged on the base 6 in order to attach the strap layer 7″ to the base. In the present embodiment the straps 7 and optionally also 7′ are formed by a hook-and-loop structure to facilitate releasable fastening of on portion of the strap to another portion of the strap. In fact, the entire strap layer 7″ could be a sheet of Velcro™ or similar hook-and-loop fastener material.

The upper surface of the plaster 4 further comprises an adhesive surface portion 8 which is adapted for retaining a fixed position of the tube.

An adhesive 14 is arranged on the strapping structure 7 to attach a foam layer 15 on top of the strapping structure 7. The foam layer 15 forms a protective layer shielding the person from the pressure of the tube (not shown in this figure).

The foam layer 15 comprises a serrated edge 15′ facilitating forming of a tapered shape of the foam layer by deformation hereof. To compress the serrated edge 15′ and thereby form the mentioned tapered shape, an edge portion 6′ of the base is folded down to cover the serrated edge 15′ of the foam 15. Simultaneously, the adhesive lower surface of this folded portion of the base thereby forms the adhesive upper surface portion 8 of the plaster and thereby facilitates better fastening of the tube 2 to the upper surface. The dotted line 23 indicated the folding line for the base 6.

A top 16 is arranged on top of the foam layer 15 and fastened by an adhesive 17. The top 16 is arranged for protection of the layers below. It should be understood, that the foam layer 15 in an alternative embodiment may be arranged on top of the top 16, thereby forming the uppermost layer of the plaster 4, and thereby at least a part of the upper surface of the plaster, as illustrated in FIG. 5b.

The plaster device 101 of FIG. 6 is similar to the plaster device 1 of FIGS. 5a and 5b. However, some of the layers are formed differently due to the smaller size of this embodiment. Furthermore, a foam layer is not present in the illustrated embodiment 101. It should however be understood that the smaller sized plaster device 101 in another embodiment may comprise a foam layer.

In the present embodiment, the strapping structure 107 is made from a non-woven material with an adhesive layer 118. The adhesive layer 118 is covered by a third protective layer 119 in two pieces. In order to securely fasten a tube after positioning hereof, the protective layer 119 is removed and the two ends of the strapping structure 107 are joined.

FIG. 7 illustrates the attachment procedure of a plaster device 1. Each of the figures, i.e. FIG. 7a-FIG. 7i, illustrates a step in the attachment procedure.

FIG. 7a—Step a: A plaster device 1 ready for use.

FIG. 7b—Step b: The protective layer 11 is removed from the lower surface of plaster 4 by grapping the tab 20.

FIG. 7c—Step c: The plaster device 1 is attached to the skin of a person by pressing the adhesive lower surface onto the skin. Subsequently, the protective layer 10 is removed from the adhesive surface portion 8.

FIG. 7d—Step d: The junction 9 of the tube 2 is pressed onto the adhesive surface portion 8.

FIG. 7e—Step e: Two of the lumens 21 of the tube 2 are fastened by the first set of straps 7A, while the third lumen 21′ is elevated to avoid fastening hereof.

FIG. 7f—Step f: All three lumens 21, 21′ are securely fastened by the second set of straps 7B.

FIG. 7g—Step g: The remaining part of the protective layer 10 is removed so that the adhesive of the cover 5 is exposed.

FIG. 7h—Step h: The cover 5 is attached to the skin so that it co-extends the plaster 4, i.e. the cover 5 is positioned in continuation of the plaster 4 so that an overlap 22 exists between the adhesive surface portion 8 and a part of the cover 5. The cover 5 is transparent to enable monitoring of the access opening 3 and the part of the tube which is below the cover 5.

FIG. 7i—Step i: The plaster device 1 and the tube 2 are securely fastened to the skin, and ready for use.

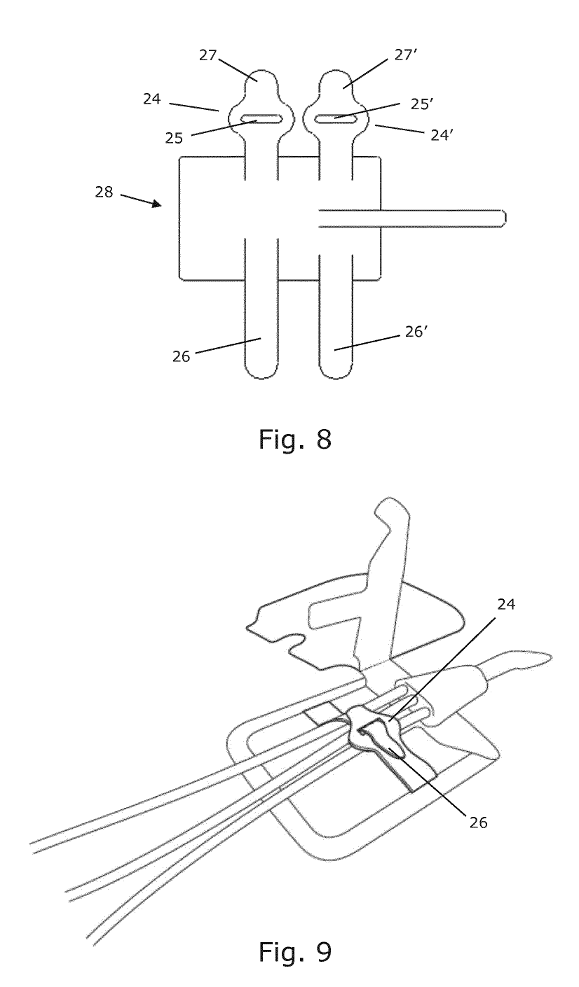

FIG. 8 illustrates a strap layer 28 in one embodiment of the device. This strap layer includes both straps 26, 26′ and head portions 27, 27′. The straps are receivable through the slots of the heads to form a closed loop configuration.

The strap layer 28 could be double-sided e.g. with adhesive on one surface or with a hook-and-loop type fastening structure, e.g. of the kind used in the plaster illustrated in FIG. 5a. When double-sided, one surface is joinable to the opposite surface to close the loop, and it becomes very easy to tighten the loop around the tube. The illustrated strap is a double strap with two matching head and tail portions 24, 24′, 26, 26′, but it could also be a single strap with a single matching head, or it could be one long strap and 2, 3, 4 or more heads such that the single strap could be inserted through several slots.

FIG. 9 illustrates the same kind of plaster illustrated in FIG. 2 and with a hook-and-loop type strap of the kind illustrated in FIG. 8. In this embodiment, the plaster includes a single strap with only one set of matching head and tail portions 24, 26. The strap could also be in the form of a binder where opposite ends of the same strap forms a head and a tail which is receivable through the slot of the head.

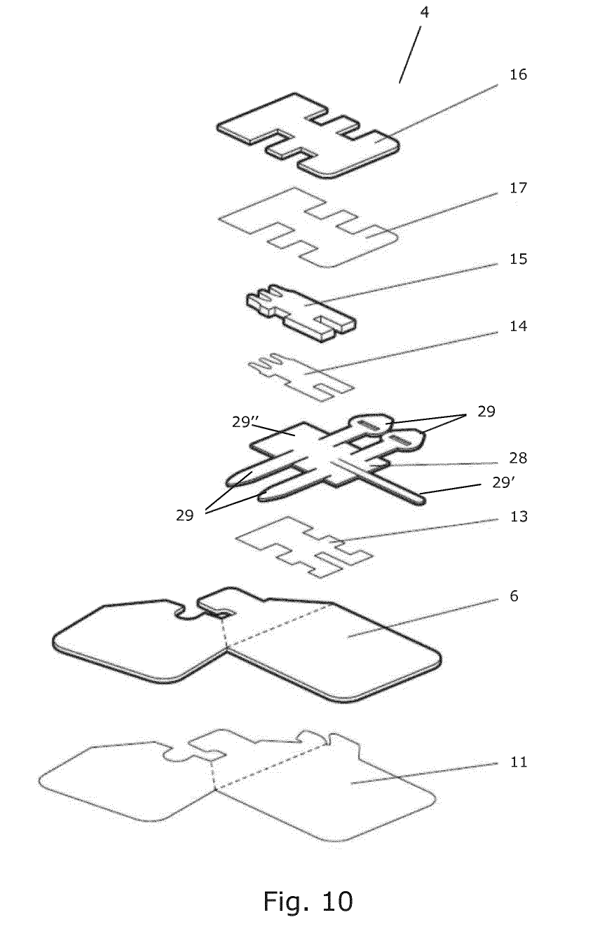

FIG. 10 illustrates an exploded view of an embodiment of the plaster device 1 of FIG. 1. All elements in the laminate structure correspond to, and are numbered identically to the elements in FIG. 5a except for the straps. The straps marked as 7, 7′ in FIG. 5a are replaced with a strap layer 28 of the kind illustrated in FIG. 8, i.e. including two straps in one piece and having matching head and tail portions 29. The shape is made by stamping or cutting out the desired shape from a piece of a sheet material.

1. A plaster device for securing a medical tube to a skin surface of an individual having the tube inserted into a body part via an access opening, the plaster device comprising a plaster having a lower surface which is at least partly adhesive and facilitates attaching the plaster device to the skin, and an opposite upper surface, the plaster device further comprising at least one strap for strapping a length of the tube to the upper surface of the plaster.

2. The device according to claim 1, where the upper surface further comprises an adhesive surface portion for retaining a fixed position of the tube relative to the upper surface.

3. The device according to claim 1, further comprising a cover which is interconnected to the plaster and has a lower surface which is at least partly adhesive and facilitates attaching the cover to the skin.

4. The device according to claim 3, where the cover is more elastically deformable than the plaster to facilitate covering of the access opening.

5. The device according to claim 3, where the cover is transparent.

6. The device according to claim 3, where a transition between the cover and the plaster forms a hinge-structure allowing rotation of the cover relative to the plaster about a straight line.

7. The device according to claim 6, where the cover, by rotation about the straight line, can arrive at an end-position where it covers the adhesive surface portion.

8. The device according to claim 7, where the cover, in the end-position, co-extends the plaster in a direction forming a longitudinal support direction for the tube and facilities covering of the access opening.

9. The device according to claim 8, where the at least one strap is adapted for strapping a tube which is arranged in the longitudinal support direction.

10. The device according to claim 3, where the cover has a smaller surface area than the plaster.

11. The device according to claim 1, comprising at least one head portion forming a slot dimensioned to receive one of the at least one straps there through.

12. The device according to claim 11, where the head portion has a first surface structure and one of the at least one straps has a second surface structure, the first and second surface structures being configured for releasable bonding of the first surface structure to the second surface structure.

13. The device according to claim 1, where a double-sided one of the at least one straps or of the at least one head portions has a first surface with a first surface structure and a second surface being opposite the first surface and having a second surface structure, the first and second surface structures being configured for cooperating engagement to enable joining of the first surface with the second surface during formation of a closed loop.

14. The device according to claim 13, where at least one double-sided strap forms in one end a head portion and in an opposite end, a tail portion, the tail portion having a width matching a width of a slot provided in the head portion such that the tail portion can be received through the slot to form the closed loop.

15. The device according to claim 13, where the strapping structure comprises a hook-and-loop structure.

16. The device according to claim 1, comprising at least two sets, each formed by two cooperating straps, the at least two sets of straps being arranged at a distance from each other.

17. The device according to claim 16, where the at least two straps are arranged at a distance in the longitudinal support direction.

18. The device according to claim 1, where the at least one strap is secured to the plaster between a base which forms the lower surface and a top which forms the upper surface.

19. The device according to claim 18, where the base and top are joined adhesively.

20. The device according to claim 1, wherein the plaster comprises a plurality of layers including a foam layer which comprises a foam material.

21. The device according to claim 20, where the foam layer has a thickness of at least half of the total thickness of the plaster.

22. The device according to claim 18, wherein the at least one strap is positioned between the foam layer and the base.

23. The device according to claim 18, where all straps are formed in one piece from a sheet material having a shape with elongate projections.

24. The device according to claim 23, where the sheet is positioned between the base and a layer of non-woven material.

25. The method of securing a medical tube to a skin surface of an individual having a tube inserted into a body part via an access opening by use of a plaster device according to claim 1, the method comprising: attaching the lower surface of the plaster device to the skin surface adjacent to the access opening, arranging the tube in a longitudinal support direction relative to the plaster device and against the upper surface of the plaster device, wrapping the strap(s) around the tube, and fixing a free end of the strap to the upper surface, to the strap itself, to a head portion, or to another strap of the same plaster device.
